SEQUENCE LISTING (36 sequences)

SEQ ID NO: 1 - DNA, 22 bp, synthetic construct
  source 1..22: mol_type = other DNA
aagctacctctcagcctacttt

SEQ ID NO: 2 - DNA, 21 bp, synthetic construct
  source 1..21: mol_type = other DNA
ccactgttttctgtacccgga

SEQ ID NO: 3 - DNA, 22 bp, synthetic construct
  source 1..22: mol_type = other DNA
tttccgcaaggttcgattttca

SEQ ID NO: 4 - DNA, 20 bp, synthetic construct
  source 1..20: mol_type = other DNA
ggcatctgcgctctaccatc

SEQ ID NO: 5 - DNA, 21 bp, synthetic construct
  source 1..21: mol_type = other DNA
aatcctccgttcctggtgtat

SEQ ID NO: 6 - DNA, 22 bp, synthetic construct
  source 1..22: mol_type = other DNA
aagactccttgtacttgggact

SEQ ID NO: 7 - DNA, 20 bp, synthetic construct
  source 1..20: mol_type = other DNA
gccatgcttagtgacaccga

SEQ ID NO: 8 - DNA, 19 bp, synthetic construct
  source 1..19: mol_type = other DNA
ggactggaggtagtaccgc

SEQ ID NO: 9 - DNA, 21 bp, synthetic construct
  source 1..21: mol_type = other DNA
ccctgggaaaacactcacaca

SEQ ID NO: 10 - DNA, 22 bp, synthetic construct
  source 1..22: mol_type = other DNA
gcacaactcaatggtactgtcg

SEQ ID NO: 11 - DNA, 19 bp, synthetic construct
  source 1..19: mol_type = other DNA
cagcggtgaatggcacttc

SEQ ID NO: 12 - DNA, 19 bp, synthetic construct
  source 1..19: mol_type = other DNA
ggcatggacttggcaggaa

SEQ ID NO: 13 - DNA, 21 bp, synthetic construct
  source 1..21: mol_type = other DNA
ctgtcctgtgtgtcagaccag

SEQ ID NO: 14 - DNA, 21 bp, synthetic construct
  source 1..21: mol_type = other DNA
tgtgggctcatcttgacctct

SEQ ID NO: 15 - DNA, 21 bp, synthetic construct
  source 1..21: mol_type = other DNA
ctcagttcctacgcttcgcat

SEQ ID NO: 16 - DNA, 20 bp, synthetic construct
  source 1..20: mol_type = other DNA
gtcgaggtcagtgaacagca

SEQ ID NO: 17 - DNA, 20 bp, synthetic construct
  source 1..20: mol_type = other DNA
ctgcccaccctcaacttctc

SEQ ID NO: 18 - DNA, 21 bp, synthetic construct
  source 1..21: mol_type = other DNA
gcaggatcgactcgtgtttgt

SEQ ID NO: 19 - DNA, 22 bp, synthetic construct
  source 1..22: mol_type = other DNA
agcgacaagatccctttcattc

SEQ ID NO: 20 - DNA, 19 bp, synthetic construct
  source 1..19: mol_type = other DNA
cgttgccggacttcacctt

SEQ ID NO: 21 - DNA, 21 bp, synthetic construct
  source 1..21: mol_type = other DNA
gtggcattcaaggagtacctc

SEQ ID NO: 22 - DNA, 22 bp, synthetic construct
  source 1..22: mol_type = other DNA
tgatggccttcgattctggatt

SEQ ID NO: 23 - DNA, 22 bp, synthetic construct
  source 1..22: mol_type = other DNA
ttattcccaatgcgttccaaga

SEQ ID NO: 24 - DNA, 23 bp, synthetic construct
  source 1..23: mol_type = other DNA
gcactaaaagactcaagcctcat

SEQ ID NO: 25 - DNA, 22 bp, synthetic construct
  source 1..22: mol_type = other DNA
cctctctctaatcagccctctg

SEQ ID NO: 26 - DNA, 21 bp, synthetic construct
  source 1..21: mol_type = other DNA
gaggacctgggagtagatgag

SEQ ID NO: 27 - DNA, 21 bp, synthetic construct
  source 1..21: mol_type = other DNA
ccacctggacgtattccactg

SEQ ID NO: 28 - DNA, 23 bp, synthetic construct
  source 1..23: mol_type = other DNA
tcgaatcaaatgactaggagggg

SEQ ID NO: 29 - DNA, 22 bp, synthetic construct
  source 1..22: mol_type = other DNA
aaaattacacgccagatttgcc

SEQ ID NO: 30 - DNA, 23 bp, synthetic construct
  source 1..23: mol_type = other DNA
ggtgtgacattactccagagttg

SEQ ID NO: 31 - DNA, 19 bp, synthetic construct
  source 1..19: mol_type = other DNA
cccgccatcggttcagttc

SEQ ID NO: 32 - DNA, 20 bp, synthetic construct
  source 1..20: mol_type = other DNA
ccccgagtaagcatgtccac

SEQ ID NO: 33 - DNA, 23 bp, synthetic construct
  source 1..23: mol_type = other DNA
caaggactacagactacgcttca

SEQ ID NO: 34 - DNA, 21 bp, synthetic construct
  source 1..21: mol_type = other DNA
tccgagggaaatggagaacct

SEQ ID NO: 35 - DNA, 20 bp, synthetic construct
  source 1..20: mol_type = other DNA
tcctcctgtttcatccaagc

SEQ ID NO: 36 - DNA, 20 bp, synthetic construct
  source 1..20: mol_type = other DNA
tagtagccgggccctacttt

(19) United States
(12) Patent Application Publication
(10) Pub. No.: US 2024/0254560 A1
(43) Pub. Date: Aug. 1, 2024

(54) Application of Feature Gene TRIM22 in Preparation of Reagent Regulating Expression of Breast Cancer-Related Gene

(71) Applicant: Cancer Institute and Hospital, Beijing (CN)

(72) Inventors: Yan WANG, Beijing (CN); Baowen YUAN, Beijing (CN); Wei HUANG, Beijing (CN); Hefen YU, Beijing (CN); Jingyao ZHANG, Beijing (CN); Yunkai YANG, Beijing (CN)

(73) Assignee: Cancer Institute and Hospital, Beijing (CN)

(21) Appl. No.: 18/429,449

(22) Filed: Feb. 1, 2024

(30) Foreign Application Priority Data
Feb. 1, 2023 (CN) 202310126050.3

(51) Int. Cl. C12Q 1/6886; C12Q 1/6851

(52) U.S. Cl. CPC C12Q 1/6886; C12Q 1/6851; C12Q 2600/112; C12Q 2600/118; C12Q 2600/158

(57) ABSTRACT

An application of feature gene TRIM22 in preparing reagent for regulating expression of breast cancer-related genes is provided. The feature gene TRIM22 disclosed in the present disclosure upregulates the expression of breast cancer-related genes SIX3, GATA6, PTX3, MMP1 and DMBT1 through overexpression, and downregulates the expression of the breast cancer-related genes SOX4, CXCL10, TNF, TP63 and CXCL16 through overexpression. Therefore, the feature gene TRIM22 provided in the present disclosure can be used as a reagent product to regulate the expression of breast cancer-related genes GATA6, SIX3, SOX4, CXCL10, PTX3, TNF, TP63, MMP1, CXCL16 and DMBT1 through overexpression.

### CROSS REFERENCE TO THE RELATED APPLICATIONS

This application is based upon and claims priority to Chinese Patent Application No. 202310126050.3, filed on Feb. 1, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC171_Sequence_Listing.xml, created on 01/26/2024, and is 32,599 bytes in size.

### TECHNICAL FIELD

The present disclosure relates to the technical field of feature genes, and particularly to an application of a feature gene TRIM22 in preparing a reagent for regulating expression of breast cancer-related genes.

### BACKGROUND

Breast cancer is the cancer with the highest morbidity and mortality among women. Globally, there were estimated 2.1 million newly diagnosed female breast cancer cases in 2018, accounting for nearly a quarter of female cancer cases. Triple-negative breast cancer (TNBC) can be diagnosed via immunohistochemical methods through the lack of amplified expression of three receptors including estrogen receptor (ER), progesterone receptor (PR), and human epidermal growth factor receptor 2 (HER2). TNBC accounts for 15-20% of all breast cancers and has a more obvious pattern of metastasis than other subtypes of the disease, with poor prognosis in patients. Many efforts have been made to understand the molecular mechanism of TNBC. The Cancer Genome Atlas (TCGA) project contributes to a comprehensive understanding of breast cancer-specific molecular heterogeneity and driver mutations, including TNBC. So far, the etiology and molecular mechanism of TNBC have not been well explained. Therefore, it is particularly important to find the prognostic biomarkers in TNBC patients and explore the molecular mechanisms underlying the high morbidity and mortality of TNBC.

TME refers to the internal and external environment of tumor cells that are closely correlated with the occurrence, growth, and metastasis of tumors. In the TME, tumor cells are able to adapt and proliferate with greatly reduced detection and eradication by host immune surveillance. In addition to tumor cells, the TME mainly includes two non-tumor components immune cells and stromal cells, which are considered to be of great significance for tumor diagnosis and prognosis. Currently, the most promising way to activate therapeutic anti-tumor immunity is blocking immune checkpoints to reduce immunosuppression. Cancer immunotherapies targeting programmed cell death protein 1 (PD-1) and programmed cell death 1 ligand 1 (PD-L1) are changing traditional tumor treatment. Moreover, anti-PD-1/PD-L1 antibodies have been proven to be of great clinical significance in more than 15 types of cancers, including melanoma, non-small cell lung cancer (NSCLC), and renal cell carcinoma (RCC). The diverse immune microenvironment in TNBC greatly influences the risk of relapse, response to chemotherapy, and applications of immunotherapy. An immune checkpoint inhibitor is now ready for use in the study of neoadjuvant and adjuvant therapies for TNBC.

ESTIMATE algorithm is a tool that uses gene expression profile characteristics to predict the proportion of stromal and immune cells in tumors, and infer the purity of tumors in tissues. Current ESTIMATE analysis has shown that stromal/immune cell in infiltration is correlated with improved prognosis in patients with various types of tumors, including glioblastoma and cutaneous melanoma. Nonetheless, no detailed analysis of TNBC immune/stromal scores is available at present.

### SUMMARY

An objective of the present disclosure is to provide an application of a feature gene TRIM22 in preparing a reagent for regulating expression of breast cancer-related genes, so as to better understand the effects of immune and stromal cell-related genes on triple-negative breast cancer (TNBC) prognosis, and uncover TME-related genes with poor prognosis to explore potential regulatory mechanisms.

In order to achieve the above objective, the present disclosure provides the following technical solutions:

The present disclosure provides an application of a feature gene TRIM22 in preparing a reagent for regulating expression of breast cancer-related genes, including the following steps:
(1) downloading a GSE21653 data set including RNA-seq data and clinicopathological information of TNBC patients from the GEO database;
(2) analyzing the GSE21653 data set using the ESTIMATE algorithm to obtain a distribution result of scores of GSE21653 samples, and dividing the distribution result of scores into high- and low-score groups based on the median;
(3) screening differentially expressed genes (DEGs) between the high- and low-score groups, and conducting survival analysis of the DEGs to obtain a gene set 1 significantly correlated with disease-free survival (DFS);
(4) analyzing the gene set 1 in KM-plotter website, and screening a gene set 2 whose gene expression levels are significantly correlated with DFS in an E-MTAB-365 data set;
(5) performing a univariate Cox regression of the gene set 2 using a R-package “survival”, and screening a gene set 3 significantly correlated with DFS according to the univariate time series result; and
(6) further analyzing the significance of the gene set 3 with DFS using multivariate Cox-LASSO regression analysis and screening feature genes correlated with prognosis of TNBC.

Preferably, functional enrichment analysis is performed on the DEGs in the step (3).

Preferably, the functional enrichment analysis includes GO enrichment analysis and KEGG pathway analysis.

Preferably, the gene set 1 is further subjected to PPI analysis, GO enrichment analysis, KEGG pathway analysis via STRING, and protein-protein interaction analysis is performed based on the PPI network module.

Preferably, a GSE58812 dataset containing clinical information of TNBC samples is further downloaded from the GEO database to verify the correlation of the screened gene set 2 with prognosis in the step (4).

Preferably, the step (5) further includes the detection of expression of the gene set 3 in normal mammary tissues and breast cancer samples using the immunohistochemical staining and the real-time RT-PCR (RT-qPCR).

The present disclosure further provides an application an application of the feature gene obtained according to the screening method as a prognostic marker for TNBC.

Preferably, the feature genes can be one or more of BIRC3, CD8A, GNLY and TRIM22.

In the present disclosure, the ESTIMATE algorithm is adopted to study the publicly available data, and multiple datasets were used to verify that the selected genes were reliable and common. The ESTIMATE algorithm is applicable to microarray expression data sets, new microarray and RNA-seq-based transcriptome profiles. In the present disclosure, the ESTIMATE algorithm is adopted to screen and obtain four feature genes BIRC3, CD8A, GNLY and TRIM22, which can be used as prognostic markers for the TNBC.

### BRIEF DESCRIPTION OF THE DRAWINGS

The application file contains at least one drawing executed in color. Copies of this patent application publication with color drawings will be provided by the Office upon request and payment of the necessary fee.

FIGS. 1A-1D show that ESTIMATE and immune scores are closely correlated with the survival of TNBC in Example 1. (FIG. 1A) Distribution of ESTIMATE, immune, and stromal scores of TNBC samples. (The violin plot shows significant correlations between the TNBC samples and the ESTIMATE, immune, and stromal scores). (FIG. 1B) The three scores of TNBC samples are divided into high- and low-score groups (taking the median as a standard). Survival analysis is performed using the clinical follow-up data corresponding to each sample. Results indicate that the ESTIMATE score is significantly correlated with the DFS of TNBC samples in the GEO database (p<0.05). (FIG. 1C) Results of correlation between high- and low-score groups of the immune score. (FIG. 1D) Results of correlation between high- and low-score groups of the stromal score.

FIG. 2 shows the identification results of differentially expressed genes (DEGs) based on the TNBC ESTIMATE score in Example 2. DEGs (|log 2FC|>1, p<0.05), each row represents a gene, and each column represents a sample; the samples are sorted according to the ESTIMATE score from high to low and from left to right, the blue group on the left is samples from the ESTIMATE high score group, and the right pink group is samples from the ESTIMATE low score group; the genes are ranked according to the p value of the differential expression analysis from low to high, red represents high gene expression and blue represents low gene expression, and the darker the red or blue color, the greater the degree of difference in gene expression.

FIG. 3 shows the top 10 molecular functions of GO annotation for all 278 differentially expressed genes (DEGs) using the STRING database in Example 2.

FIG. 4 shows the top 10 biological processes of GO annotation for all 278 DEGs using the STRING database in Example 2.

FIG. 5 shows the top 10 cellular components of GO annotation for all 278 DEGs using the STRING database in Example 2.

FIG. 6 shows the top 10 enrichment results of KEGG pathway enrichment on all 278 DEGs using the STRING database in Example 2.

FIGS. 7A-7F show that survival curves for 6 representative genes of the 171 DEGs correlated with TNBC prognosis in Example 2 are significantly correlated with DFS (p<0.05).

FIG. 8 shows a PPI network of 171 DFS-related genes constructed using the STRING tool in Example 2, and it contains a total of 145 nodes and 1438 edges. The color of a node in the PPI network reflects the log (FC) value and the size of the node represents the degree. The thickness of the edge reflects the comprehensive score of the degree of interaction between the nodes.

FIG. 9 shows the top 12 molecular functions of GO annotation of the PPI network using the STRING database in Example 2.

FIG. 10 shows the top 10 biological processes of GO annotation of the PPI network using the STRING database in Example 2.

FIG. 11 shows the top 10 cellular components of GO annotation of the PPI network using the STRING database in Example 2.

FIG. 12 shows the top 10 enrichment results of KEGG pathway enrichment of the PPI network using the STRING database in Example 2.

FIG. 13 shows the correlation of 171 DFS-related genes with TNBC prognosis in Example 2. The color of a node in the PPI network reflects the log (FC) value and the size of the node represents the degree. The thickness of the edge reflects the comprehensive score of the degree of interaction between the nodes. Module 1 of the PPI network contains 24 nodes and 245 edges.

FIG. 14 shows the correlation of 171 DFS-related genes with TNBC prognosis in Example 2. Module 2 of the PPI network contains 20 nodes and 70 edges.

FIG. 15 shows the correlation of 171 DFS-related genes with TNBC prognosis in Example 2. Module 3 of the PPI network contains 20 nodes and 64 edges.

FIG. 16 shows the correlation of 171 DFS-related genes with TNBC prognosis in Example 2. Module 4 of the PPI network contains 12 nodes and 28 edges.

FIG. 17 shows the relationship between immune microenvironment-related genes and prognosis of TNBC patients, and Kaplan-Meier survival analysis of the relationship between survival time and 14 genes expression signatures by R package survival in Example 2.

FIGS. 18A-18C show the verification of immune microenvironment-related genes in TNBC. (FIG. 18A) Immunohistochemical staining of BIRC3, CASP1, CD8A, EOMES and TRIM22 in normal breast tissues and breast carcinoma samples. (FIG. 18B) RNA expression of genes with prognostic values between normal mammary tissues and TNBC in The Human Protein Atlas database. num (N): normal sample size; Tumor (T): tumor sample size. *** p<0.001 by a two-tailed unpaired t test. (FIG. 18C) The expression levels of BIRC3, CASP1, CLIC2, EOMES, GZMB, IL2RB, and TRIM22 are measured by RT-qPCR in MCF-10A, MDA-MB-231 and Hs 578T cells. The mRNA levels are normalized to GAPDH. Error bars represent the means±standard deviations of three independent experiments (**p<0.01, ***p<0.001, two-tailed unpaired t test).

FIG. 19 is the multivariate Cox-LASSO regression analysis of the correlation between immune microenvironment-associated genes and disease-free survival.

FIG. 20 is ROC curves, showing the ability of four immune microenvironment-associated genes to predict time-dependent disease-free survival rate.

FIG. 21 is a heat map showing changes in expression profile of the cell line MDA-MB-231 after Vector and overexpression of TRIM22.

FIGS. 22A-22B show KEGG pathway enrichment analysis of differentially expressed genes.

FIG. 23 shows GSEA gene set enrichment analysis revealing both upregulated and downregulated pathways.

FIGS. 24A-24G are GSEA enrichment profiles of 7 pathways: immune system (FIG. 24A), NOTCH signaling pathway (FIG. 24B), immune effector (FIG. 24C), cell proliferation (FIG. 24D), cell response to cytokines (FIG. 24E), epithelial cell differentiation (FIG. 24F), and response to tumor necrosis factor (FIG. 24G).

FIG. 25 shows RT-qPCR analysis of selected differential genes in Vector and the cell line MDA-MB-231 after overexpression of TRIM22.

### DETAILED DESCRIPTION OF THE EMBODIMENTS

The technical solution provided in the present disclosure is described in detail below in conjunction with the examples, but they are not to be construed as limiting the scope of protection of the present disclosure.

### Example 1

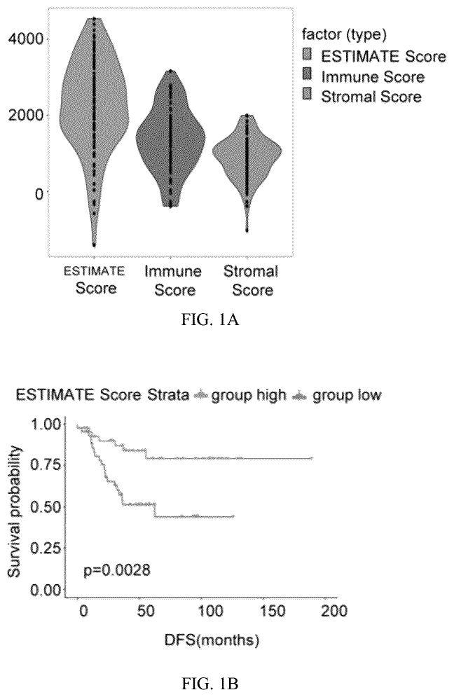

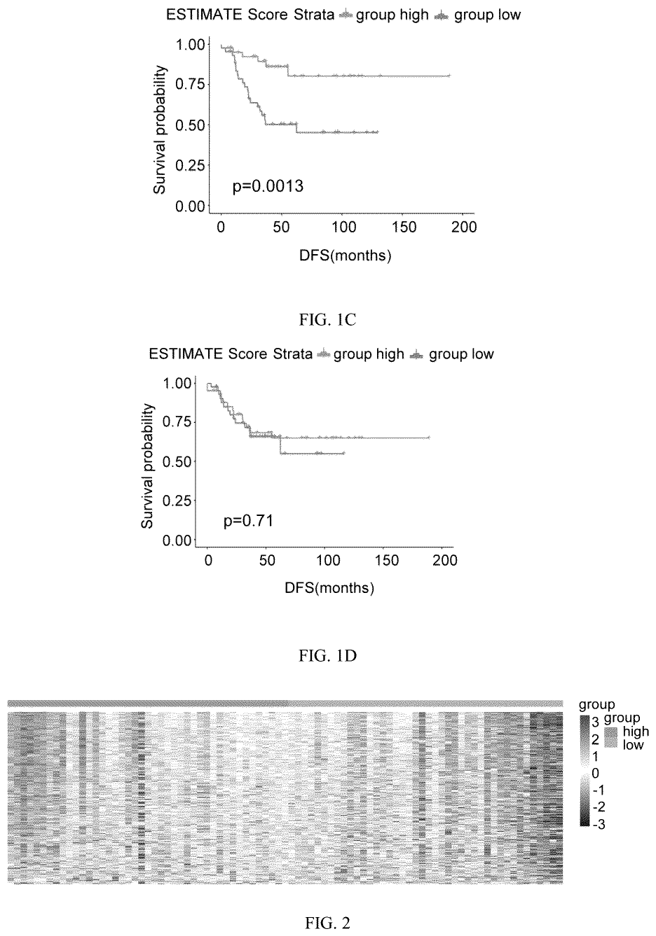

The clinical information of TNBC samples (GSE21653) was downloaded from the GEO database (https://www.ncbi.nlm.nih.gov/geo/). The samples in GSE21653 were analyzed using ESTIMATE, immune and stromal scores, respectively. The distribution of scores of the samples was shown in FIG. 1A. The TNBC samples (GSE21653) were divided into high- and low-score groups based on the median, and a survival analysis was conducted using the clinical follow-up information corresponding to the three scores for each group. The survival curve showed that the high score group of the ESTIMATE score had a higher survival rate than the low score group (p=0.0028 in log-rank test), indicating the ESTIMATE score was significantly correlated with disease-free survival (DFS) of TNBC samples from the GEO database (p<0.05) (FIG. 1B). Similar phenomena were observed in the high- and low-score groups of the immune and stromal scores (FIGS. 1C-1D).

Since the ESTIMATE score is a comprehensive evaluation of the immune and stromal scores, the genes correlated with TNBC prognosis were further explored based on the ESTIMATE score.

### Example 2

Differentially expressed genes (DEGs) were screened between the high- and low-score groups according to the ESTIMATE score groups in Example 1. Differential expression analysis was performed using the R package limma (Version: 3.42.2). The screening conditions for DEGs were |log 2FC (fold change)|>1 and FDR<0.05. A total of 278 DEGs were identified. The DEGs in the high- and low-score groups were shown in FIG. 2.

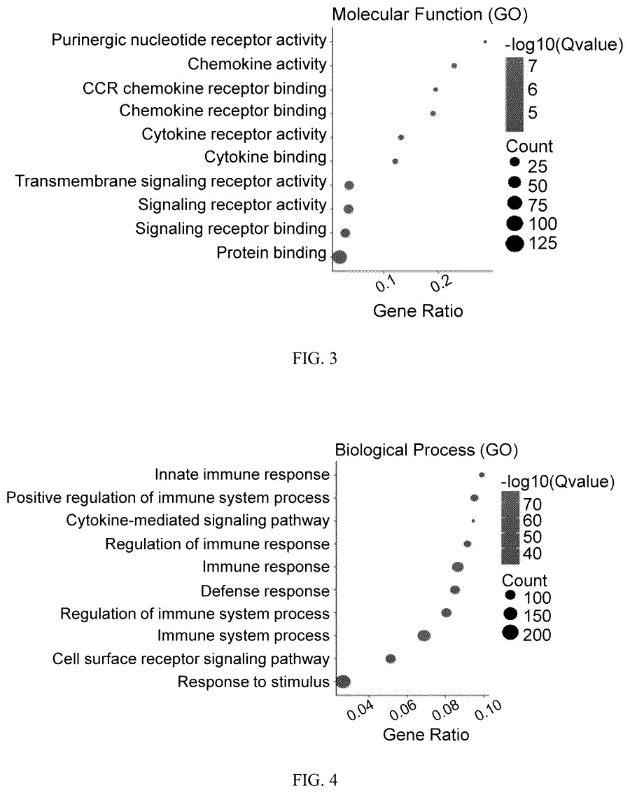

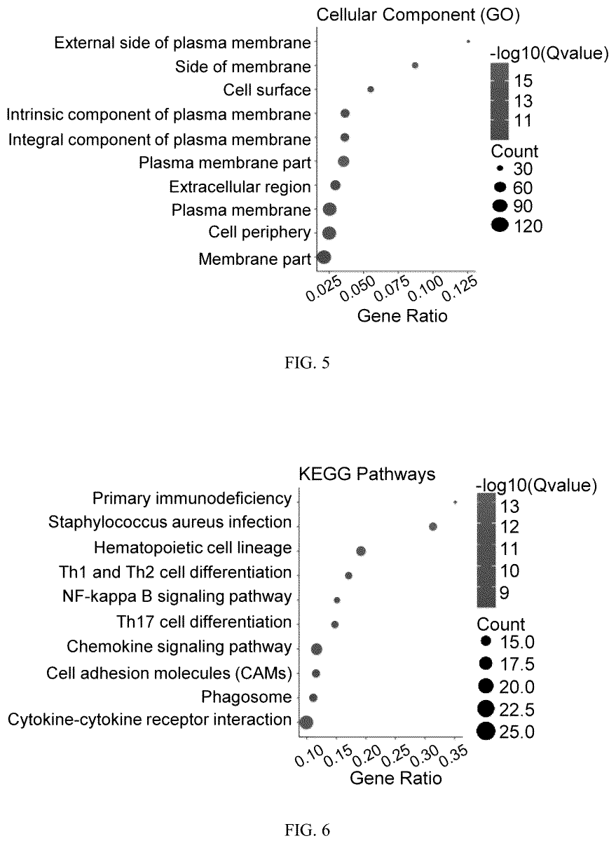

In order to study the function of these DEGs, functional enrichment analysis was performed on 276 upregulated and 2 downregulated genes via the STRING database, including GO (molecular function, biological process and cellular component) and KEGG pathway analyses. The top ten enrichment terms for each section of GO and KEGG pathways were shown in FIGS. 3-6 (sorted by −log 10 of Q value). GO functions indicated that these genes are mainly enriched in protein binding, immune system process and immune response and membrane part (FIGS. 3-5). Moreover, cytokine-cytokine receptor interaction and chemokine signaling pathway were also obtained from KEGG pathway analysis (FIG. 6).

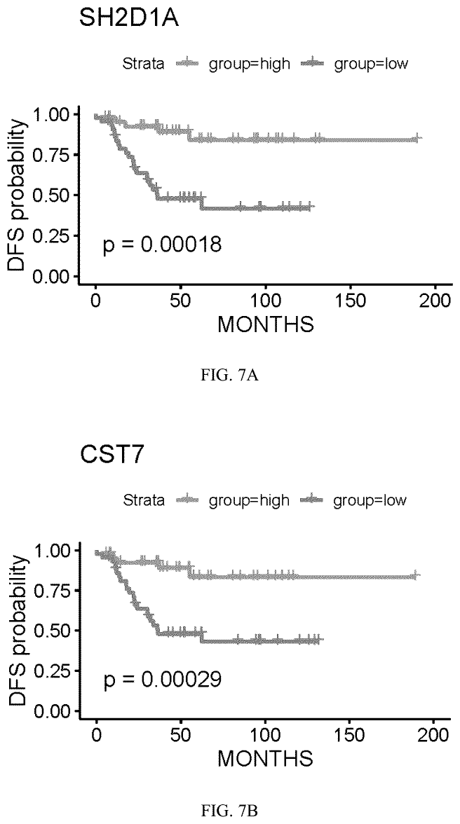

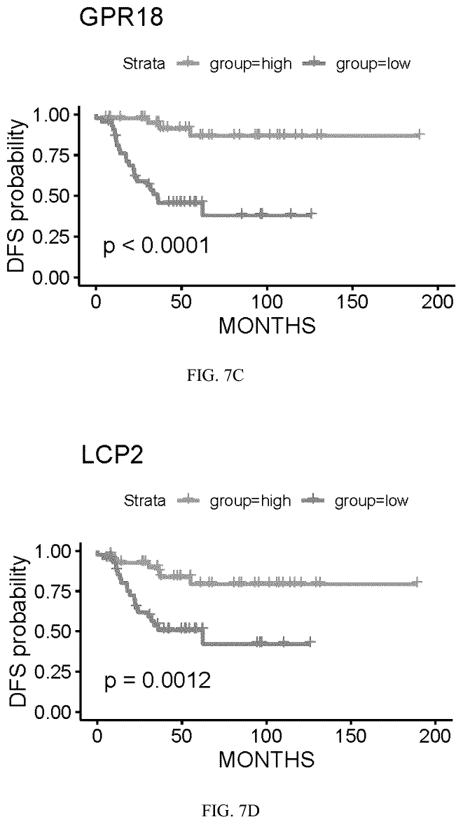

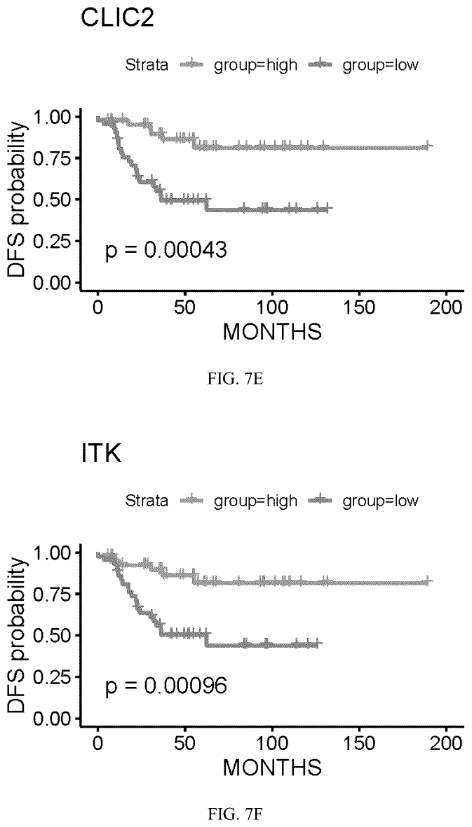

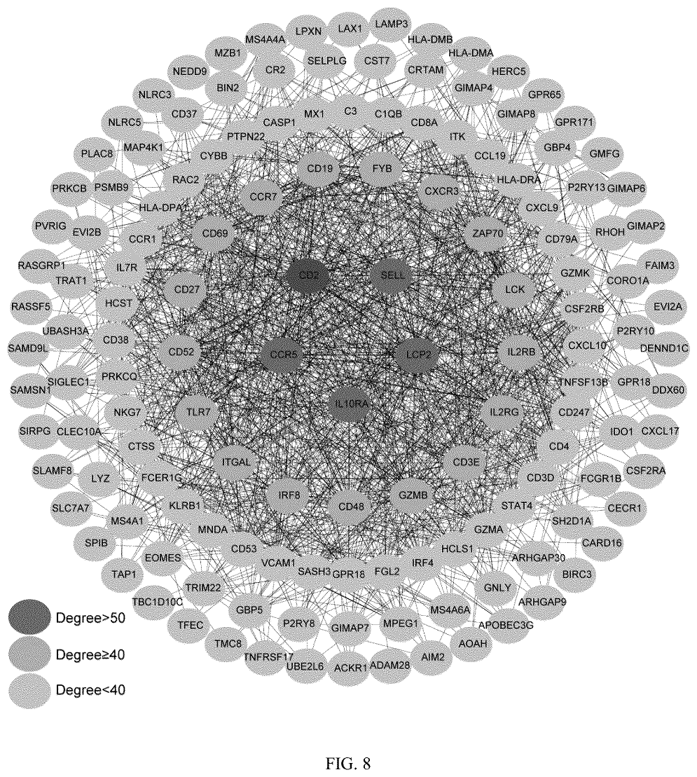

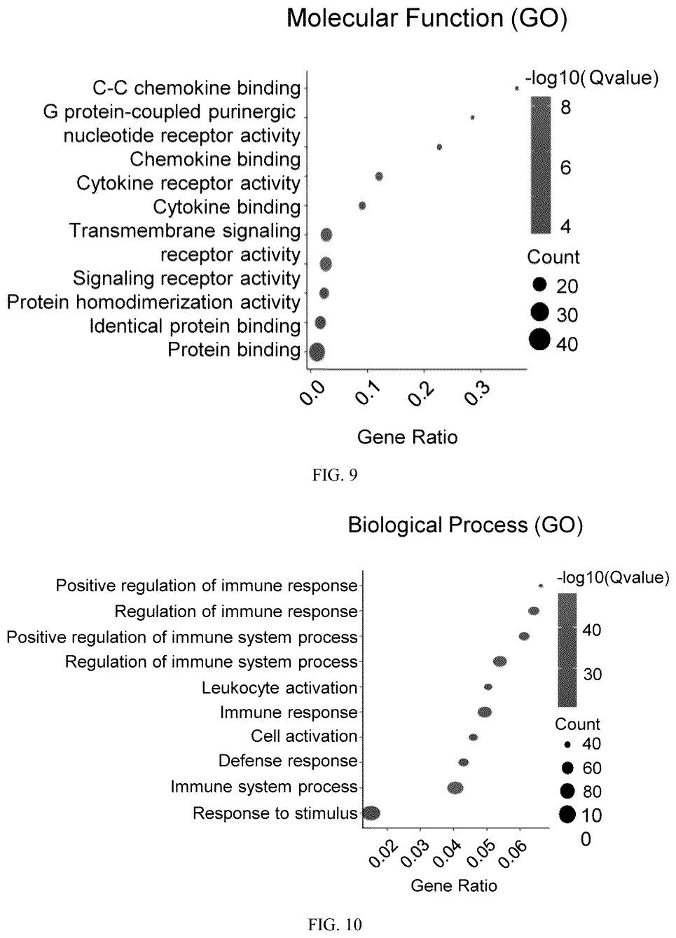

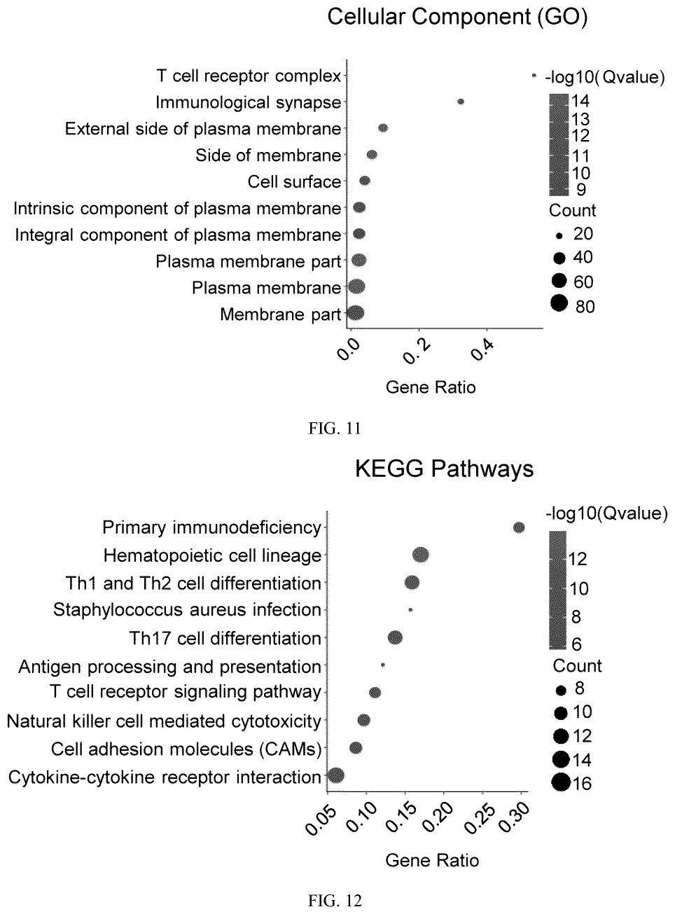

In order to screen genes correlated with prognosis of TNBC, survival analysis of all the DEGs was performed, among which 171 genes were significantly correlated with DFS (p<0.05). The survival curves of six genes with the lowest p values, including SH2D1A, CST7, GPR18, LCP2, CLIC2 and ITK, from the 171 genes were shown in FIGS. 7A-7F. The core proteins included CD2, SELL, CCR5, IL10RA and LCP2 (FIG. 8). Subsequently, the GO enrichment and KEGG pathway analyses on the genes mined by the survival analysis were carried out. The data showed these genes were mainly enriched in the TME and immune-related pathway (FIGS. 9-12).

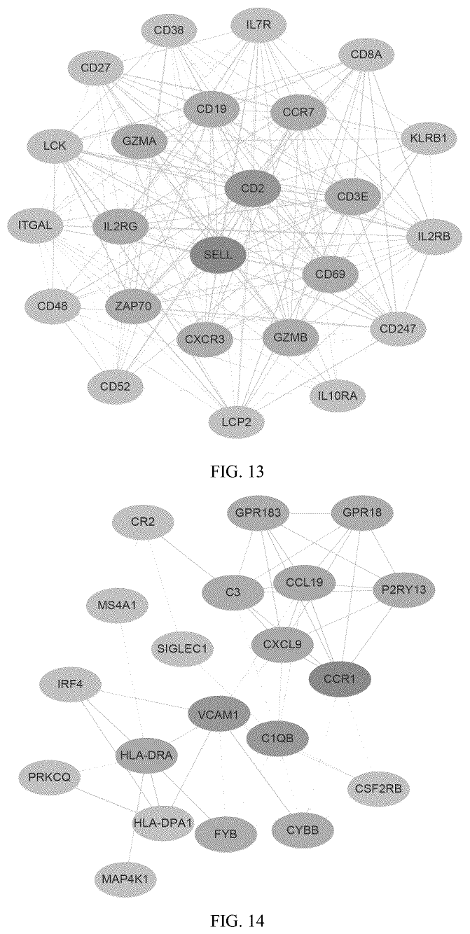

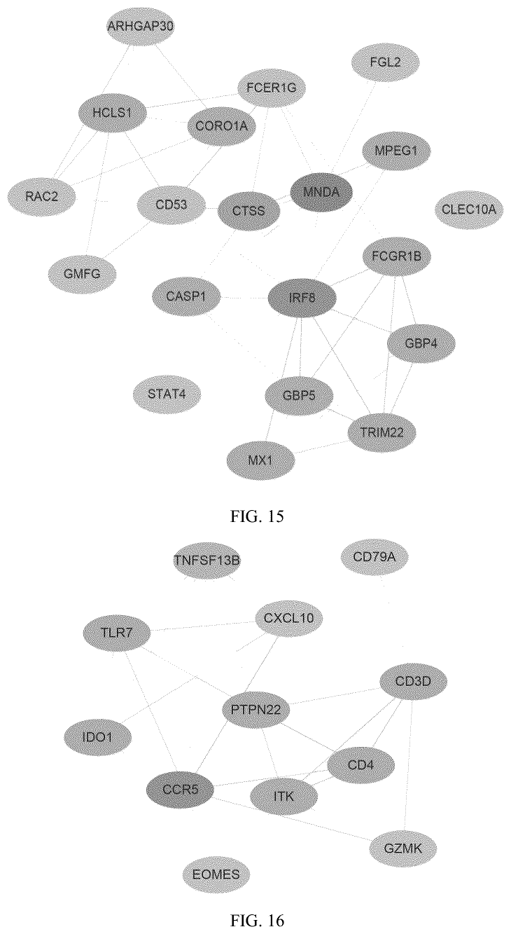

The protein interaction of the 171 DFS-related genes from the PPI network module was integrated via MCODE analysis, and the top four of the eight modules obtained were selected for further investigation. The protein interaction of these four modules with the core nodes had higher degrees. Module 1 contained a total of 24 nodes and 245 edges (FIG. 13). Among them, SELL, ITGAL, CD8A, CD52, and CD2 have been confirmed as cell adhesion markers, which are involved in a series of important physiological and pathological processes, such as immune response, tumor metastasis, and wound healing (hsa04514). Module 2 contained a total of 20 nodes and 70 edges (FIG. 14). Among them, C1QB, HLA-DRA, C3, and HLA-DPA1 were confirmed to be correlated with S. aureus infection and systemiclupus erythematosus, indicating that these genes are closely related to immune response (hsa05150, hsa05322). Module 3 contained a total of 20 nodes and 64 edges (FIG. 15). Among them, CASP1, GBP4, and GBP5 were confirmed to be correlated with Nod-like receptor signaling pathway, which was an important way for eukaryotes to recognize pathogens (hsa04621). Module 4 contained a total of 12 nodes and 28 edges, four (CD4, CCR5, CD3D and ITK) of which were reported to be closely correlated with immune response (hsa04658, hsa04060, hsa04660). As shown in FIGS. 13-16, SELL, ITGAL, CD69 and HLA-DRA had higher node degrees, indicating that they might be important immune microenvironment-related genes of TNBC. Among these 171 DFS-related genes, 11 genes were reported to be significantly correlated with breast cancer prognosis, including CD3D, CD8A, CORO1A, GZMB, LCK, TRBC1, HLA-DRA, ACSL5, EOMES, IRF4 and IRF8. In addition, CD3D, CD8A, CORO1A, GZMB, EOMES and IRF8 were reported to be correlated with the prognosis of TNBC. The association of the remaining genes, including CD247, CD3E, LAX1, LPXN, PRKCB and SIRPG, with TNBC prognosis, were not reported. These genes might be potential immune microenvironment-related prognostic markers of TNBC.

The E-MTAB-365 dataset from KM-plotter website (http://kmplot.com/analysis/index.php?p=service&cancer-breast), containing 48 TNBC samples, was used to further analyze the 171 DFS-related genes, and the expression levels of 36 genes were significantly correlated with DFS in the E-MTAB-365 dataset (p<0.01). Details of the 36 genes were shown in Table 1.

TABLE 1
Genes significantly correlated with disease-free survival
in the GSE21653 dataset and verified by KM plotter

| Category | Gene symbols |
|---|---|
| T cell receptor complex | CD247, *CD3D and *CD8A |
| Immunological synapse | CD3E, *CORO1A, *GZMB and LCK |
| Plasma membrane | LAX1, LPXN, PRKCB, SIRPG, TRBC1 |
| | and TRAC |
| Cell surface | CD19, CD27, HLA-DRA, IL2RB, SELL |
| Cell-cell junction | CRTAM, ITK, LCP2 |
| Cell membrane part | ACSL5, BIRC3, CLIC2 |
| Cytoplasmic vesicle | AOAH, CTSS, GNLY, GPR18 |
| Cell part | CASP1, CST7, *EOMES, IRF4, *IRF8, |
| | MZB1, SH2D1A, TRIM22 |

Note:
The genes in normal font are reported genes related to breast cancer prognosis; the genes marked with an asterisk (*) are reported genes related to TNBC prognosis; and the genes in bold font have not been reported to be related to breast cancer prognosis.

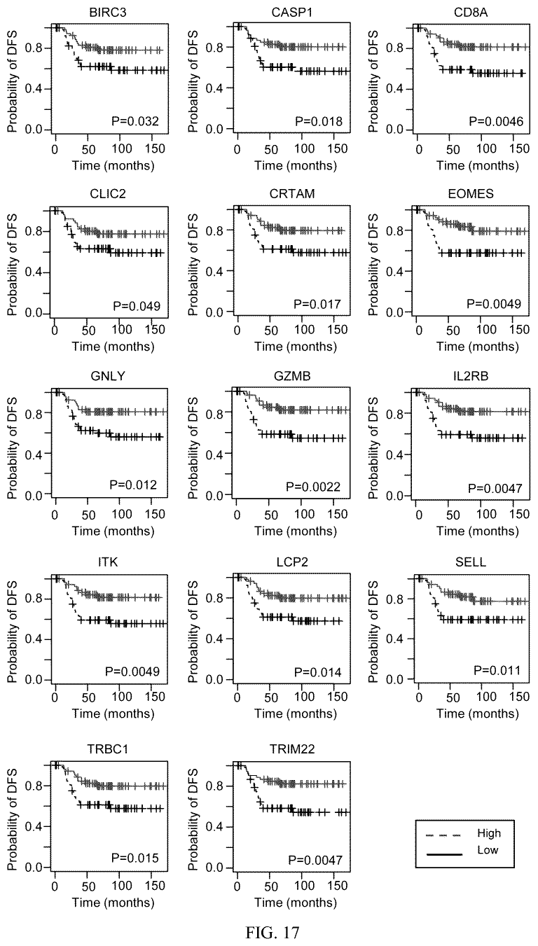

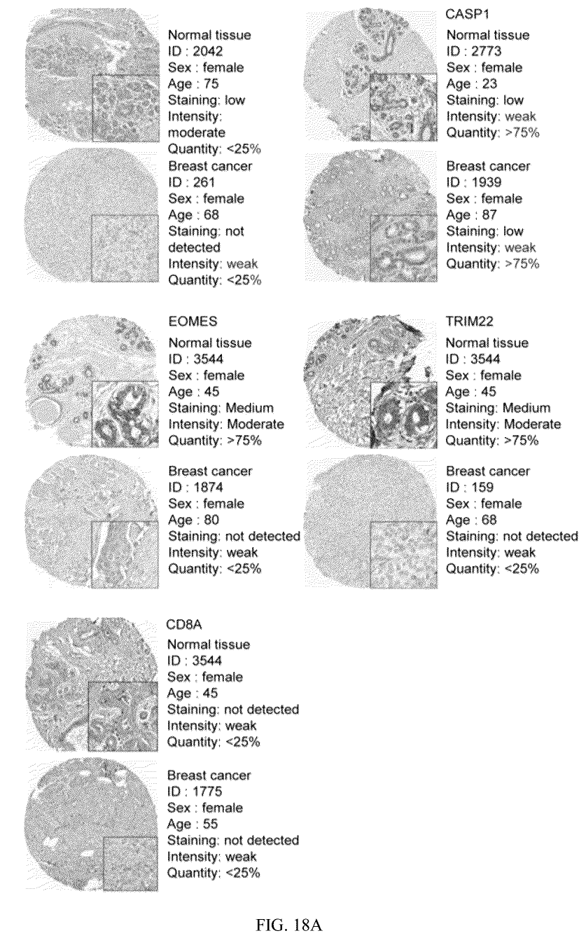

In addition, GSE58812 dataset containing clinical information of TNBC samples were downloaded from the GEO database and were used to verify the correlation of the above 36 genes with prognosis of TNBC samples. These genes were further analyzed by univariate Cox regression using the R-package “survival”. A univariate time series result indicated that only 14 genes were significantly correlated with DFS. Details of the 14 genes were shown in Table 2. Kaplan-Meier survival analysis showed that patients with higher expression of these genes have better disease-free survival (FIG. 17). Among these genes, SELL, GZMB, IL2RB, LCP2 and CD8A were involved in Module 1, CASP1 and TRIM22 were involved in Module 3, and EOMES and ITK were involved in Module 4. These genes may be potential genes for the poor prognosis of TNBC and may provide therapeutic value for TNBC in the future.

TABLE 2
Genes significantly correlated with survival in
the GSE58812 dataset and verified by R package

| Gene | | |
|---|---|---|
| symbol | Gene ID | Biological process (GO) |
| BIRC3 | 330 | GO: 0001959 regulation of cytokine- |
| | | mediated signaling pathway |
| CASP1 | 834 | GO: 0065009 regulation of molecular |
| | | function |
| CD8A | 925 | GO: 0007165 signal transduction |
| CLIC2 | 1193 | GO: 0003824 catalytic activity |
| EOMES | 8320 | GO: 0010467 gene expression |
| GNLY | 10578 | GO: 0050896 response to stimulus |
| GZMB | 3002 | GO: 0007165 signal transduction |
| ITK | 3702 | GO: 0071704 organic substance metabolic |
| | | process |
| LCP2 | 3937 | GO: 0035592 establishment of protein |
| | | localization to extracellular region |
| SELL | 6402 | GO: 0010033 response to organic substance |
| CRTAM | 56253 | GO: 0008104 protein localization |
| IL2RB | 3560 | GO: 0019899 enzyme binding |
| TRBC1 | 28639 | GO: 0050776 regulation of immune response |
| TRIM22 | 10346 | GO: 0009894 regulation of catabolic process |

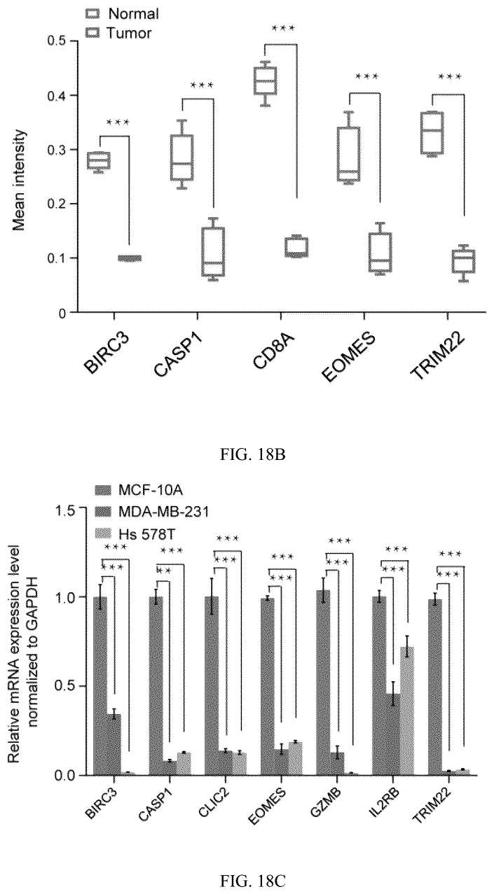

In order to further confirm the reliability of these immune microenvironment-related genes in TNBC prognosis, the expression patterns of some genes were verified. Firstly, immunohistochemical staining was used to detect the expression of 14 genes in normal mammary tissues and breast cancer samples from The Human Protein Atlas website. Results showed that BIRC3, CASP1, CD8A, EOMES and TRIM22 were significantly downregulated in breast carcinoma samples (FIGS. 18A-18B). In addition, the relative mRNA expression levels of these 14 genes were detected by real-time RT-PCR (RT-qPCR) in MCF-10A, MDA-MB-231 and Hs 578T cells. The results showed that BIRC3, CASP1, CLIC2, EOMES, GZMB, IL2RB, and TRIM22 were notably downregulated in MDA-MB-231 and Hs 578T cells compared with MCF-10A cells (FIG. 18C). These immune microenvironment-related genes might be good prognostic biomarkers of TNBC.

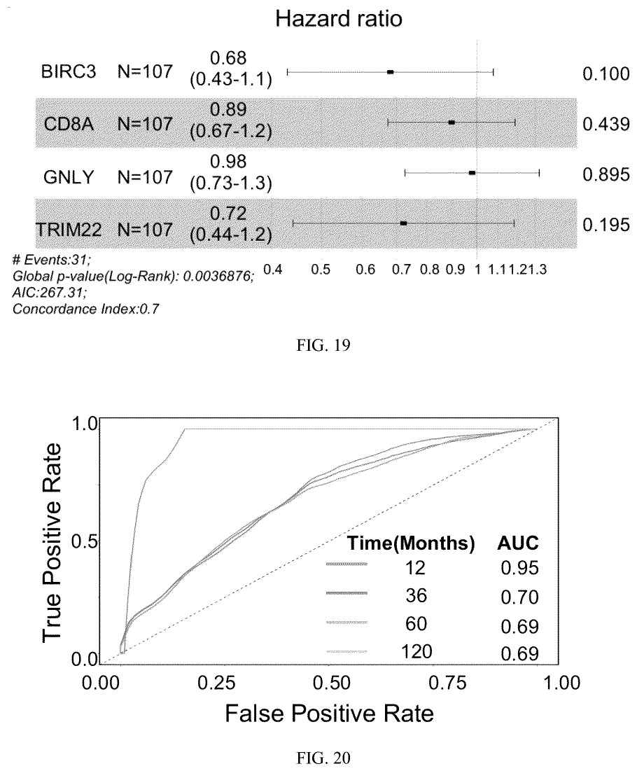

Multivariate Cox-LASSO regression analysis was used to further validate the significance of the 14 genes. BIRC3 (hazard ratio, 0.68; 95% CI, 0.43-1.1; p=0.1), CD8A (hazard ratio, 0.89; 95% CI, 0.67-1.2; p=0.439), GNLY (hazard ratio, 0.98; 95% CI, 0.73-1.3; p=0.895), and TRIM22 (hazard ratio, 0.72; 95% CI, 0.44-1.2; p=0.195) may be closely correlated with the prognosis (FIG. 19). Subsequently, a time-dependent receiver operating characteristic (ROC) curve of these four genes for the prognosis was completed. An AUC value for predicting one-year survival rate was 0.95 (FIG. 20). Overall, these four immune microenvironment-related genes can be used as feature genes for TNBC prognosis (the NCBI number of the nucleotide sequence of the BIRC3 gene is Gene ID: 330, the NCBI number of the nucleotide sequence of the CD8A gene is Gene ID: 925, the NCBI number of the nucleotide sequence of the GNLY gene is Gene ID: 10578, and the NCBI number of the nucleotide sequence of the GNLY gene is Gene ID: 10346).

In order to determine how TRIM22 regulates the development of breast cancer, RNA sequencing (RNA-seq) experiments were performed in MDA-MB-231 cells infected with Vector and FLAG-TRIM22 lentiviruses, respectively.

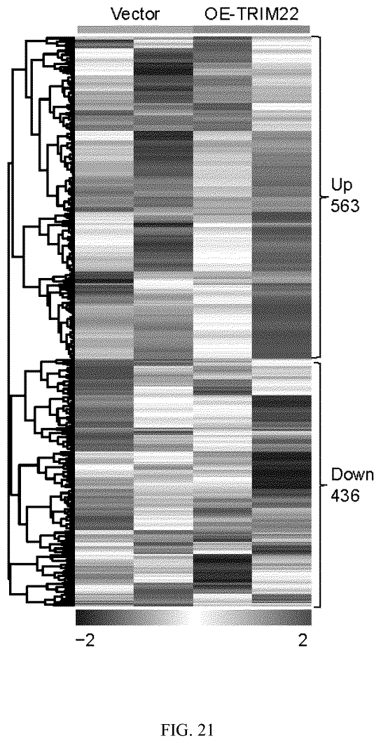

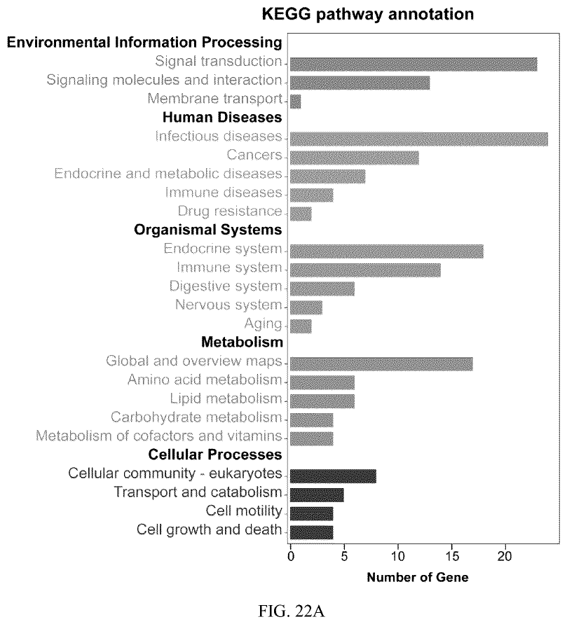

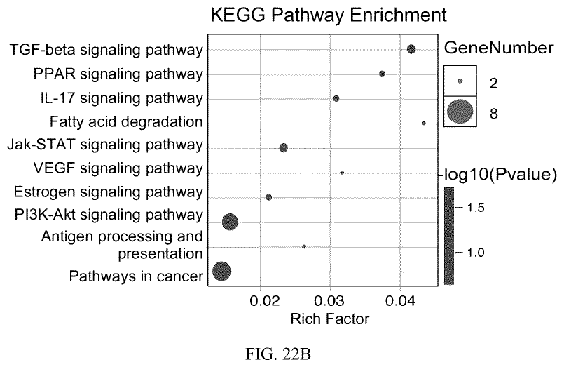

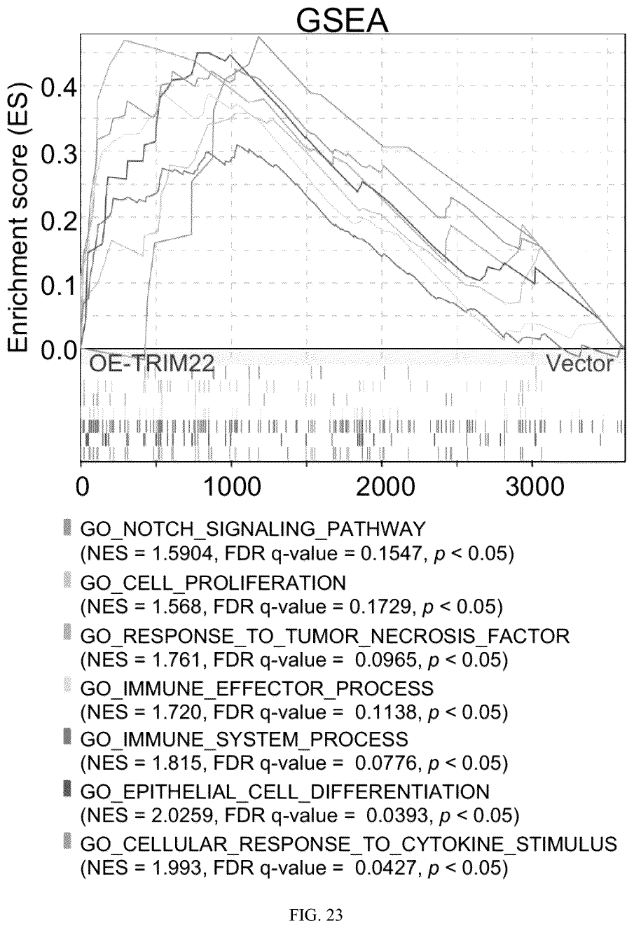

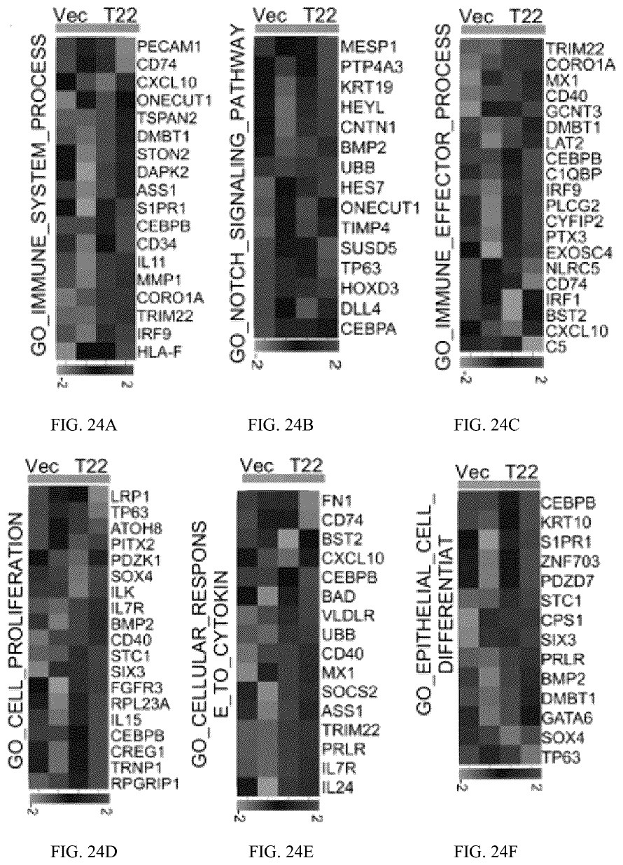

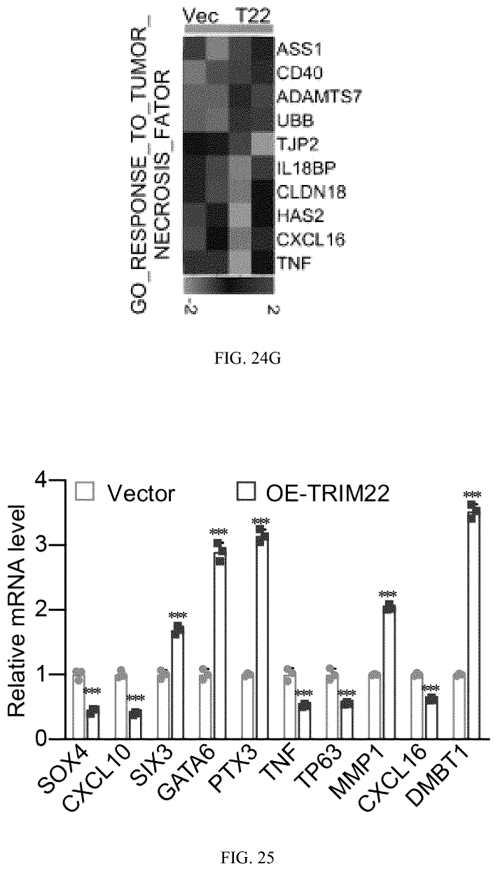

Compared with Vector-infected controls, 563 upregulated genes and 436 downregulated genes were identified in TRIM22 overexpressing cells (FIG. 21). Differentially expressed genes by KEGG analysis (http://www.kegg.jp/kegg/pathway.html) showed that differentially expressed genes are enriched in pathways of environmental information processing, human diseases, metabolism, cellular processes including TGF-β, fatty acid degradation, carcinogenic-related immune microenvironment activities (FIGS. 22A-22B). Gene set enrichment analysis (GSEA) of differentially expressed target genes revealed that GO enriched harvested NOTCH signal, cell proliferation, response to tumor necrosis factor, immune effector processes, immune system processes, epithelial cell differentiation, cell response to cytokine stimulation (FIG. 23). Further analysis of differentially expressed target genes in the biological process (GO-BP) were shown in FIGS. 24A-24G.

Then, 10 representative genes correlated with carcinogenesis that play a role in the interaction of tumor cells and the immune system (including GATA6, SIX3, SOX4, CXCL10, PTX3, TNF, TP63, MMP1, CXCL16 and DMBT1) were selected, and response of these genes to overexpression of TRIM22 in MDA-MB-231 cells was verified by RT-qPCR (FIG. 25).

In the above example, protein-protein interaction (PPI) analysis refers to PIP analysis conducted via STRING. Then PPI network was reconstructed using Cytoscape software, which showed a network of more than 10 nodes. The node size was related to the node degree, with the thickness of the edges reflecting a score of the degree of interaction between the nodes, and the color of the nodes reflecting the differential expression degree. The MCODE tool of Cytoscape was used for PPI network analysis and showed the PPI modules of the DEGs.

Functional and pathway enrichment analysis referred to the functional enrichment analysis of DEGs via the STRING, including GO, KEGG pathways, Reactome pathways, UniProt keywords, PFAM protein domains, INTERPRO protein domains and features, and SMART protein domains. The GO analysis contained molecular functions (MF), biological processes (BP), and cellular components (CC).

Immunohistochemical staining images were downloaded from The Human Protein Atlas (https://www.proteinatlas.org/). RNA expression of genes with prognostic values between normal tissues and TNBC were presented by GraphPad Prism (https://www.graphpad.com/).

The cell lines used in cell culture were obtained from the American Type Culture Collection. MCF-10A cells were cultured with the mammary epithelium growth factor medium (MEGM) kit supplemented with growth factors (Lonza). Hs 578T cells were cultured with the Dulbecco's Modified Eagle Medium (DMEM). Cells were maintained in a humidified incubator equilibrated with 5% CO₂ at 37° C. MDA-MB-231 cells were cultured in L-15 medium without CO₂. In addition to MEGM, all the other media were supplemented with 10% fetal bovine serum (FBS), 100 units/ml penicillin, and 100 mg/ml streptomycin (Gibco).

In the RT-qPCR process, total RNA was extracted with Trizol reagent according to the manufacturer's instructions (Invitrogen). Potential DNA contamination was avoided with RNase-free DNase treatment (Promega). cDNA was prepared with MMLV Reverse Transcriptase (Roche). Relative quantitation of gene expression was performed using the ABI PRISM 7500 sequence detection system (Applied Biosystems) measuring real-time SYBR green fluorescence. The results were acquired using the comparative Ct method (2-ΔΔCt) with GAPDH as an internal control. This experiment was performed at least thrice independently. The primer sequences used were listed in Table 3.

TABLE 3
Primer sequences used in RT-qPCR

| Gene | Chain | Sequence | SEQ ID NO: |
|---|---|---|---|
| BIRC3 | F | AAGCTACCTCTCAGCCTACTTT | 1 |
| BIRC3 | R | CCACTGTTTTCTGTACCCGGA | 2 |
| CASP1 | F | TTTCCGCAAGGTTCGATTTTCA | 3 |
| CASP1 | R | GGCATCTGCGCTCTACCATC | 4 |
| CLIC2 | F | AATCCTCCGTTCCTGGTGTAT | 5 |
| CLIC2 | R | AAGACTCCTTGTACTTGGGACT | 6 |
| EOMES | F | GCCATGCTTAGTGACACCGA | 7 |
| EOMES | R | GGACTGGAGGTAGTACCGC | 8 |
| GZMB | F | CCCTGGGAAAACACTCACACA | 9 |
| GZMB | R | GCACAACTCAATGGTACTGTCG | 10 |
| ILR2B | F | CAGCGGTGAATGGCACTTC | 11 |
| ILR2B | R | GGCATGGACTTGGCAGGAA | 12 |
| TRIM22 | F | CTGTCCTGTGTGTCAGACCAG | 13 |
| TRIM22 | R | TGTGGGCTCATCTTGACCTCT | 14 |
| GATA6 | F | CTCAGTTCCTACGCTTCGCAT | 15 |
| GATA6 | R | GTCGAGGTCAGTGAACAGCA | 16 |
| SIX3 | F | CTGCCCACCCTCAACTTCTC | 17 |
| SIX3 | R | GCAGGATCGACTCGTGTTTGT | 18 |
| SOX4 | F | AGCGACAAGATCCCTTTCATTC | 19 |
| SOX4 | R | CGTTGCCGGACTTCACCTT | 20 |
| CXCL10 | F | GTGGCATTCAAGGAGTACCTC | 21 |
| CXCL10 | R | TGATGGCCTTCGATTCTGGATT | 22 |
| PTX3 | F | TTATTCCCAATGCGTTCCAAGA | 23 |
| PTX3 | R | GCACTAAAAGACTCAAGCCTCAT | 24 |
| TNF | F | CCTCTCTCTAATCAGCCCTCTG | 25 |
| TNF | R | GAGGACCTGGGAGTAGATGAG | 26 |
| TP63 | F | CCACCTGGACGTATTCCACTG | 27 |
| TP63 | R | TCGAATCAAATGACTAGGAGGGG | 28 |
| MMP1 | R | AAAATTACACGCCAGATTTGCC | 29 |
| MMP1 | F | GGTGTGACATTACTCCAGAGTTG | 30 |
| CXCL16 | R | CCCGCCATCGGTTCAGTTC | 31 |
| CXCL16 | F | CCCCGAGTAAGCATGTCCAC | 32 |
| DMBT1 | R | CAAGGACTACAGACTACGCTTCA | 33 |
| DMBT1 | F | TCCGAGGGAAATGGAGAACCT | 34 |
| GAPDH | R | TCCTCCTGTTTCATCCAAGC | 35 |
| GAPDH | F | TAGTAGCCGGGCCCTACTTT | 36 |

The statistical analysis process was to fit Ten-fold cross-validated Cox Survival Analysis and Least Absolute Shrinkage and Selection Operator Regression (Cox-LASSO regression) model, as implemented in R package glmnet and survival. The corresponding hazard ratio (HR), 95% confidence interval (CI), and p value were collected. A forest plot was drawn using the R package survminer. Time-dependent receiver operating characteristic (ROC) curves were carried out to estimate the predictive accuracy for prognosis and describe the associated AUCs for the years of 1-10 based on the risk score by the R package pROC. From the curves, sensitivity, specificity, likelihood ratio, predictive value and their respective 95% confidence intervals can be seen. Herein p-value <0.05 were considered statistically significant.

In the present disclosure, the ESTIMATE algorithm was adopted to study the publicly available data, and multiple datasets were used to verify that the selected genes were reliable and common. The “CIBERSORT” method in the prior art was adopted to analyze microarray data, but not for TCGA RNA-seq data. Since the “TIMER” method has limited sample size and relevance, it is unable to distinguish the location of immune cells in the stroma or tumor or to capture tumor cell heterogeneity. The ESTIMATE algorithm is applicable to microarray expression data sets, new microarray and RNA-seq-based transcriptome profiles. The predictive ability of this method has been verified in large independent data sets. Findings of the present disclosure is helpful for people to gain a better understanding of the complex regulatory network of TNBC, and the functions of immune and stromal cell-related genes in the progression of TNBC. These findings may provide new promising biomarkers for the treatment of TNBC.

The feature genes BIRC3, CD8A, GNLY and TRIM22 screened in the present disclosure can be used as prognostic markers for the TNBC. BIRC3 (baculoviral IAP repeat containing 3) participates in immunity activities by regulating NF-κB signaling and other in ammatory signals. It also acts as an E3 ubiquitin protein ligase in the TME in mice. The TNFa-TNFR2-BIRC3-TRAF1 signaling pathway has been shown to promote metastases in mice, and the activation of this pathway is correlated with the poor prognosis of gastrointestinal stromal tumor patients.

CD8A (CD8 antigen) is a cell surface glycoprotein on most cytotoxic T lymphocytes, which mediates effective cell-to-cell interactions in the immune system. It functions as a coreceptor with T cell receptors on T lymphocytes and recognizes antigens displayed by antigen-presenting cells in class I MHC molecules. As stated earlier, CD8A was predictive for increased pathologic complete response (pCR) in the neoadjuvant GeparSixto trial. The CD8A gene is correlated with an improved outcome in several public breast cancer datasets.

GNLY (granulysin) is part of the saposin-like protein (SAPLIP) family and is located in the cytotoxic granules of T cells and released after antigen stimulation. GNLY may tempt ER stress-mediated apoptosis. It is correlated to the ability of NK-extracellular vesicles (EV) to induce cytotoxicity. In addition, serum GNLY may be a potential biomarker for nasopharyngeal carcinoma, the early stage of colorectal adenocarcinoma and muscle-invasive bladder cancer. The function of GNLY in TNBC immune microenvironment and immunotherapies deserves further research.

TRIM22 (Stimulated Trans-Acting Factor of 50 kDa, Staf-50) is an E3 ubiquitin-ligase and a member of the C-IV group of tripartite motif (TRIM) family, which is strongly induced by interferon stimulation and takes part in innate immunity of cells. Apart from the antiviral effects, TRIM22 is a potential therapeutic target and prognosis marker for NSCLC. Further exploration of TRIM22 in TNBC cells also revealed its carcinogenesis-related role in the interaction between tumor cells and the immune system (FIGS. 7A-7F). The expression and function of TRIM22 in TNBC will be further explored.

The description above is merely the preferred embodiments of the present disclosure, it should be pointed out that those of ordinary skill in the art can also make some improvements and modifications without departing from the principle of the present disclosure, and these improvements and modifications should also fall within the scope of protection of the present disclosure.

What is claimed is:

1. An application of a characteristic gene TRIM22 in preparing a reagent for regulating expression of breast cancer-related genes, wherein the characteristic gene regulates the expression of breast cancer-related genes SIX3, GATA6, PTX3, MMP1, and DMBT1 through overexpression, and downregulates the expression of the breast cancer-related genes SOX4, CXCL10, TNF, TP63, and CXCL16 through overexpression.
